(19) United States
(12) Patent Application Publication
(10) Pub. No.: US 2022/0176163 A1
(43) Pub. Date: Jun. 9, 2022

(54) FLEXIBLE AND WEARABLE LONG DURATION ULTRASOUND DEVICE

(71) Applicant: ZetrOZ Systems LLC, Trumbull, CT (US)

(72) Inventor: George K. LEWIS, Jr., Trumbull, CT (US)

(73) Assignee: ZetrOZ Systems LLC, Trumbull, CT (US)

(21) Appl. No.: 17/271,181

(22) PCT Filed: Aug. 26, 2019
(86) PCT No.: PCT/US2019/048212

§ 371 Date: Feb. 24, 2021

Related U.S. Application Data
(60) Provisional application No. 62/722,867, filed on Aug. 25, 2018.

(51) Int. Cl. A61N 7/00; B06B 1/06

(52) U.S. Cl. CPC A61N 7/00; B06B 1/0611; A61N 2007/0017; G10K 11/30; A61N 2007/0078; A61N 2007/006; A61N 2007/0021

(57) ABSTRACT

The present invention generally relates to, inter alia, devices, methods, and systems for use in medical applications for humans and animals involving long duration ultrasound treatment. An ultrasound transducer array is provided that includes (i) a plurality of ultrasound transducers arranged in a matrix formation and operably coupled to an electrical network and (ii) a mesh structure securing the plurality of ultrasound transducers in the matrix formation, where each ultrasound transducer is connected to the electrical network in a manner sufficient to allow each ultrasound transducer to operate independent of one another or in unison. Ultrasound systems including the ultrasound transducer array and methods of using the ultrasound systems are also disclosed.

### CROSS-REFERENCE TO RELATED APPLICATIONS

This application is a U.S. National Phase filing under 35 U.S.C. § 371 of International Application No. PCT/US2019/048212, filed Aug. 26, 2019, and published as WO 2020/046847 A1 on Mar. 5, 2020, which claims priority benefit of U.S. Provisional Patent Application Ser. No. 62/722,867, filed Aug. 25, 2018, the disclosures of which are hereby incorporated by reference herein in their entirety.

### FIELD OF THE INVENTION

The present invention generally relates to, inter alia, devices, methods, and systems for use in medical applications involving long duration ultrasound treatment.

### BACKGROUND OF THE INVENTION

Ultrasound has been used to manage and treat pain and injury to tissue of subjects, as well as to heal wounds. However, there continues to be a need for improved ultrasound treatments that involve long-duration ultrasound delivery to the tissue of subjects in need of pain management and healing of tissue and wounds.

The present invention is directed to overcoming these and other deficiencies in the art.

### SUMMARY OF THE INVENTION

The present invention generally relates to, inter alia, devices, methods, and systems for use in medical applications involving long duration ultrasound treatment. More particularly, the present disclosure provides an ultrasound transducer array device, systems that include the device, and methods of using the device. The ultrasound transducer array device is flexible, wearable, and suitable for use in medical applications involving long duration ultrasound treatment, including without limitation, pain management, treatment of soft tissue injuries, and wound healing.

In one aspect, the present invention relates to an ultrasound transducer array comprising: (i) a plurality of ultrasound transducers arranged in a matrix formation and operably coupled to an electrical network; and (ii) a mesh structure securing the plurality of ultrasound transducers in the matrix formation, where each ultrasound transducer is connected to the electrical network in a manner sufficient to allow each ultrasound transducer to operate independent of one another or in unison.

In another aspect, the present invention relates to an ultrasound system comprising: (i) an ultrasound transducer array according to the present disclosure; and (ii) a power source operably connected to the ultrasound transducer array.

In another aspect, the present invention relates to a method of applying ultrasound energy to a surface of a subject. This method involves: (i) providing an ultrasound system according to the present disclosure; and (ii) applying therapeutic ultrasound energy to a subject, where the therapeutic ultrasound energy is generated by the ultrasound system.

Without intending to limit the scope thereof of the present disclosure, provided below are some features of certain embodiments of the disclosed ultrasound transducer arrays, ultrasound systems, and methods of the present disclosure.

For example, the array of individual transducers can be wired together in a redundant configuration and mechanically connected through a mesh-structure (e.g., plastic, polymer, metal, etc.) to allow for both electrical and mechanical robustness and flexibility. The mesh designs allow for air/liquid/breathability of the device on the skin and mechanical strength of the device. The wiring is flexible and extendable so that the device may conform to various configurations on the body and stretch in various planes. The wiring does not limit special orientation of the transducer elements, this is limited by the mesh structure. The low-profile and flexibility of the device allows for attachment to the body for multiple days without being obtrusive or creating pressure spots on the body. The flexible ultrasound transducer of the device may be cut to fit various contours of the body. The device may be as large or as small as desired with any number of elements. No lenses or focusing lenses or dispersing lenses or a combination of all can be used to define a specific pattern in the body. In one embodiment the individual transducer elements are epoxy-connected to the mesh-structure to create a continuous/flexible device that can stretch in various dimensions to contour to the patient.

In another configuration the elements can be connected through a silicone network and wired with flexible PCBA and/or wires/cables. The mesh structure also may act as a support system for hydrogel to be filled into the mesh and around the active ultrasound elements, thereby providing coupling of the ultrasound to the patient. The mesh structure provides a means to secure the device to the patient with adhesive bandage.

The flexible ultrasound transducer may have a closely integrated circuit to provide driving of the device, which may be connected to a separate ultrasound applicator. The flexible ultrasound transducer may have a means to measure temperature of each element with embedded thermocouple to provide safety and prevent overheating.

The ultrasound transducer array and ultrasound system can be used for various medical applications, including, for example, for pain/soft tissue healing, as well as for wound healing.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1 illustrates one embodiment of an ultrasound transducer array of the present disclosure.

FIG. 2 illustrates another embodiment of an ultrasound transducer array of the present disclosure.

FIG. 3 illustrates another embodiment of an ultrasound transducer array of the present disclosure.

FIG. 4 illustrates another embodiment of an ultrasound transducer array of the present disclosure.

FIG. 5 illustrates another embodiment of an ultrasound transducer array of the present disclosure.

FIG. 6 illustrates one embodiment of an ultrasound system of the present disclosure.

FIG. 7 is a schematic of a flow chart showing aspects of an ultrasound system of the present disclosure.

FIG. 8 is a schematic of a flow chart showing aspects of an ultrasound system of the present disclosure.

FIG. 9 is a photo of one embodiment of an ultrasound transducer array of the present disclosure.

FIG. 10 is a schematic illustrating three different embodiments of the ultrasound transducer array of the present disclosure.

FIGS. 11A-11D are photos of an embodiment of an ultrasound transducer array of the present disclosure and how it is manufactured.

FIGS. 12A-12D are photos of an embodiment of an ultrasound transducer array of the present disclosure and how it is used.

FIGS. 13A-13D are illustrations of an embodiment of an ultrasound system of the present disclosure and how it is packaged and used.

FIG. 14 is a photo the front and back of an embodiment of an ultrasound transducer array of the present disclosure.

FIGS. 15A-15B are photos of an embodiment of an ultrasound transducer array of the present disclosure and how it is manufactured.

FIGS. 16A-16B are schematics of circuit diagrams of an embodiment of an ultrasound transducer array and ultrasound system of the present disclosure.

FIG. 17 is a photo of an embodiment of an ultrasound system of the present disclosure.

FIGS. 18A-18B are photos of an embodiment of an ultrasound transducer array of the present disclosure.

FIGS. 19A-19B are photos of an embodiment of an ultrasound transducer array of the present disclosure.

FIG. 20 is a graph showing results of wound healing experiments using an embodiment of an ultrasound transducer array and ultrasound system of the present disclosure.

### DETAILED DESCRIPTION OF THE INVENTION

The present disclosure relates to an ultrasound transducer array, as further described herein. The present disclosure also relates to various ultrasound kits and ultrasound systems configured to include the ultrasound transducer array of the present disclosure. Further, the present disclosure relates to various methods of using and making the ultrasound transducer array and ultrasound system of the present disclosure.

The ultrasound transducer array and ultrasound system the present disclosure have various attributes, as described more fully herein. Without meaning to limit the present disclosure to a particular embodiment, provided below are various attributes of the present disclosure.

In one aspect, the present disclosure provides an ultrasound transducer array comprising: (i) a plurality of ultrasound transducers arranged in a matrix formation and operably coupled to an electrical network; and (ii) a mesh structure securing the plurality of ultrasound transducers in the matrix formation, where each ultrasound transducer is connected to the electrical network in a manner sufficient to allow each ultrasound transducer to operate independent of one another or in unison.

In certain embodiments, the ultrasound transducer array is configured to be flexible and wearable, thereby being suitable for long duration ultrasound application to a subject. As used herein, a “subject” can include a human or an animal. The term “patient” may also be used herein interchangeably with the term “subject.”

In certain embodiments, the matrix formation includes the plurality of ultrasound transducers oriented in substantially the same plane with their ultrasound emitting surfaces facing the same direction.

A suitable matrix formation can include, without limitation, an m-by-n matrix. However, the matrix formation need not be in the form of an m-by-n matrix. Further, the matrix formation can be such that the individual ultrasound transducers of the array are in rows and columns that contain either the same or different number of ultrasound transducers per row or per column. Further, the ultrasound transducers need not be in a straight alignment, but may be staggered.

In certain embodiments, the electrical network is configured to have redundant wiring. Suitable examples of redundant wiring can include, without limitation, redundant wiring that is configured so that removal of one or more individual ultrasound transducer from the array does not affect operability of the remaining ultrasound transducers of the array.

In certain embodiments, the electrical network is configured to have redundant wiring with each transducer in parallel to minimize electrical impedance of the transducer array.

In certain embodiments, the electrical network is configured with primary power distribution to one or more individual ultrasound transducer along a single line that said primary power distribution is protected with redundant wiring at each terminating edge of the transducer array so that removal of one or more individual ultrasound transducer from the array does not affect operability of the remaining ultrasound transducers of the array.

In certain embodiments, the electrical network is configured with a parallel crossing and grid power distribution to provide electrical redundancy and minimize electrical impedance by connecting each individual transducer in parallel.

In certain embodiments, the electrical network includes electrical components that can include, without limitation, electrical wires, coaxial cable, flexible printed circuit board (PCB), a flexible circuit, or combinations thereof.

In certain embodiments, the electrical components are configured to include additional relief sufficient to allow the array to conform to a desired shape, such as the shape of the treatment surface of a subject.

In certain embodiments, the ultrasound transducer array includes a plurality of ultrasound transducers connected via flexible circuitry and laminated between two waterproof flexible sheets.

In certain embodiments, the ultrasound transducer array includes a plurality of ultrasound transducers embedded in a silicone structure and connected via a flex circuit.

In certain embodiments, the ultrasound transducer array includes a plurality of ultrasound transducers embedded in silicone and connected with wires.

In certain embodiments, the ultrasound transducer array includes a plurality of ultrasound transducers connected via conductive ink.

In certain embodiments, the mesh structure includes a mechanical mesh material that can include, without limitation, nylon, metal, polymer, silicone, plastic, fibers, a plant-derived compound, or a combination thereof.

In certain embodiments, the ultrasound transducers of the transducer array are configured as low-profile transducers.

In certain embodiments, the ultrasound transducers of the transducer array are configured as low-profile ultrasound transducers having one or more of the following attributes: (i) a frequency of between about 250 kHz to about 4 MHz; (ii) a thickness of less than 1 cm; and/or (iii) power output capability of 0-3 Watts, and intensity of 20 mW/cm²-10 W/cm².

In certain embodiments, the ultrasound transducer array is capable of 0-100 Watts of power output, intensity 20 mW/cm²-10 W/cm².

In certain embodiments, the ultrasound transducer array has an electrical input impedance of less than 1 ohm, less than 0.25 ohm, or less than 0.01 ohm.

In certain embodiments, the ultrasound transducers are single crystal type transducers. More particularly, in certain embodiments, the ultrasound transducers are single crystal type transducers made of a lead zirconate titanate (PZT) material.

In certain embodiments, the ultrasound transducers can have a shape that includes, without limitation, the shape of a disk, rectangle, triangle, square, oval, and any other geometric shape.

In certain embodiments, the ultrasound transducers are multiple crystal design transducers. More particularly, in certain embodiments, the ultrasound transducers are multiple crystal design transducers including piezoelectric stacks in parallel for low-frequency and low electrical impedance for ultrasound power production in a flexible form.

In certain embodiments, the multiple crystal design transducers are in a form of a stack sufficient to provide low electrical impedance and excitation voltage for the ultrasound transducer.

In certain embodiments, the ultrasound transducers further include a lens that can include, without limitation, a convex lens, a concave lens, and/or a flat lens. In certain embodiments, the lens can be made from materials such as, but not limited to, TPX, Ultem, Rexolite, and metal.

In certain embodiments, the lens of the ultrasound transducer is backed with epoxy containing bubbles to isolate the lens and minimize ultrasound transmission in an unintended direction (e.g., a direction that is not directed to a treatment surface of a subject).

In certain embodiments, the ultrasound transducers are air-backed with a thermocouple embedded into a housing sufficient to enable monitoring of the temperature of the ultrasound transducers during operation.

In certain embodiments, the ultrasound transducers and electrical network are combined in the matrix formation in a manner sufficient to seal the ultrasound transducers and electrical network so as to prevent electrical shorting. For example, in certain embodiments, the ultrasound transducers and electrical network are secured to the mesh structure with a connection medium that can include, without limitation, epoxy, glue, welding, magnetic, adhesive tape, compression fit, and the like.

In certain embodiments, the ultrasound transducers are secured to the mesh structure by a back-seal comprising a mixture of epoxy and microbubbles.

In certain embodiments, the ultrasound transducer array can further include a hydrogel coated to the ultrasound emitting face of the ultrasound transducer array and mesh structure to enable ultrasonic coupling between the array and a subject contacting surface.

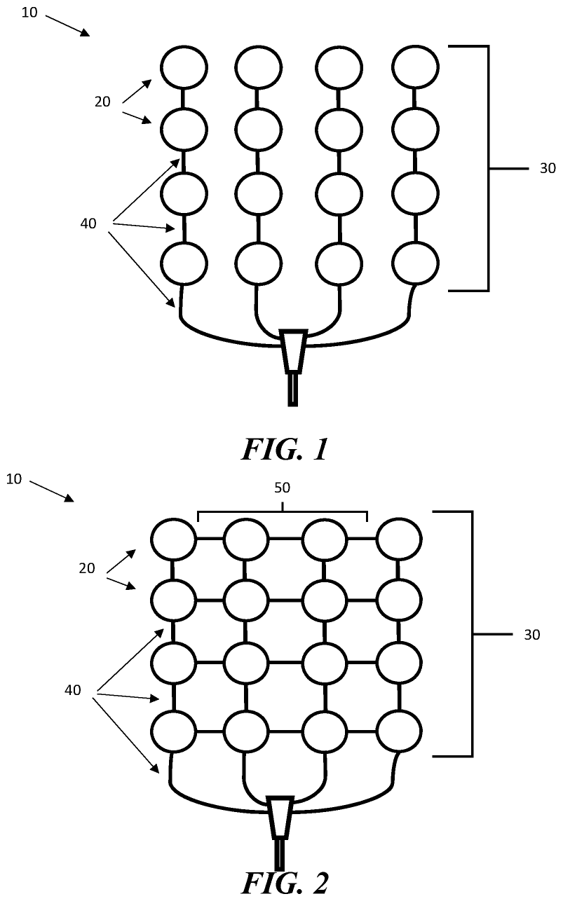

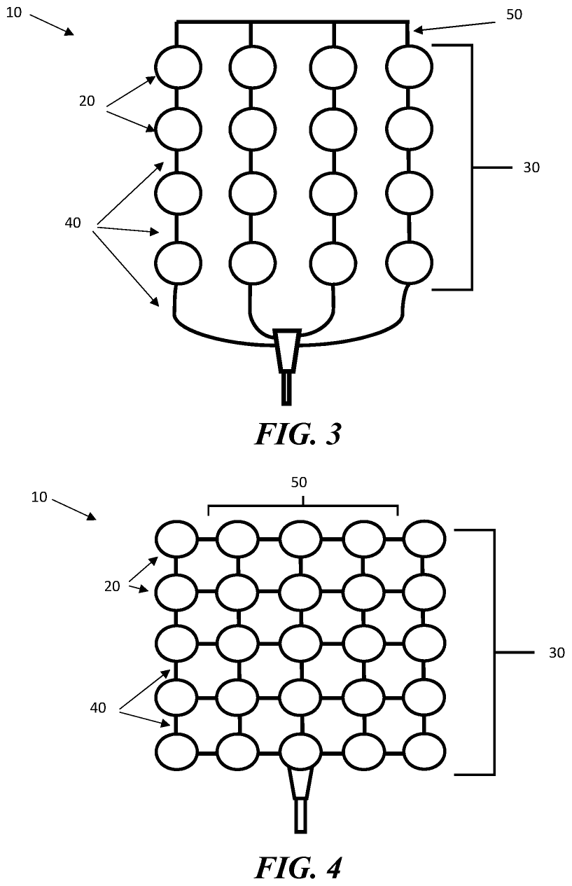

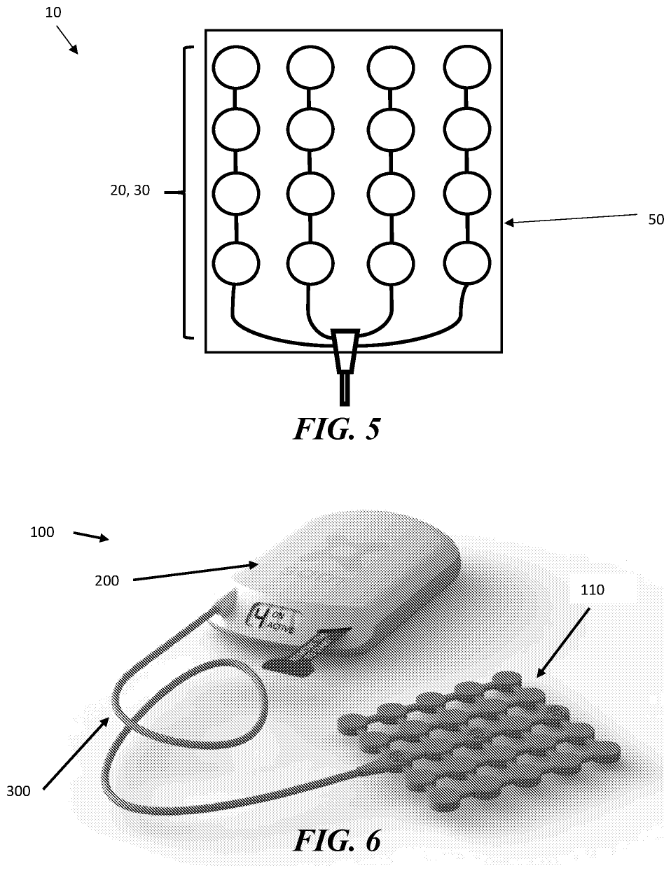

FIGS. 1-5 illustrate various features of the ultrasound transducer array of the present disclosure. With respect to FIGS. 1-5, the reference numbers of the ultrasound transducer array are identified in the paragraph below, as follows:

As shown in FIGS. 1-5, ultrasound transducer array 10 includes: (i) a plurality of ultrasound transducers 20 arranged in a matrix formation 30 and operably coupled to an electrical network 40; and (ii) a mesh structure 50 securing the plurality of ultrasound transducers 20 in the matrix formation 30, where each ultrasound transducer 20 is connected to the electrical network 40 in a manner sufficient to allow each ultrasound transducer 20 to operate independent of one another or in unison.

In another aspect, the present disclosure provides an ultrasound system comprising: (i) an ultrasound transducer array according to the present disclosure; and (ii) a power source operably connected to the ultrasound transducer array.

In certain embodiments, the ultrasound transducer array is connected to the power source with a flexible cable.

In certain embodiments, the ultrasound transducer array is powered by a power source that can include, without limitation, external power or a battery pack.

In certain embodiments, the power source is a battery. Therefore, in certain embodiments, the ultrasound transducer array is battery powered.

In certain embodiments, the power source includes components effective to provide functions that include, without limitation, power output, timing, treatment logging, dossing measurements, or any other features suitable of controlling, monitoring, or powering the ultrasound transducer array for its intended use.

In certain embodiments, the power source is a power controller device that provides energy to the ultrasound transducer array. In a particular embodiment, the power controller device includes, without limitation, the following: (i) a battery; (ii) an ultrasound transducer array driving circuitry; (iii) an on/off control (e.g., switch, button, etc.); and (iv) treatment duration increase and treatment duration decrease controls (e.g., switches, buttons).

In certain embodiments, the power source (power controller device) is configured for carrying with a belt clip.

In certain embodiments, the power source (power controller device) is configured for carrying with an arm band.

In certain embodiments, the power source (power controller device) is configured for carrying with a hook-and-loop strap.

In certain embodiments, the power source (power controller device) does not contain the driving circuitry, and the driving circuitry is held in a separate case.

In a particular embodiment, the power source is configured as a power pack that controls on/off functionality for the ultrasound transducer array and is supported by internal batteries. This power pack provides control of electrical energy to excite the ultrasound transducer array (e.g., a flexible wearable ultrasound transducer array), and also provides timing, treatment logging, dossing measurements, power-output, and confirms transducer type to be connected to the power pack.

In certain embodiments, the ultrasound system further includes a gel material (e.g., a gel or hydrogel component) for placement between the ultrasound array and a treatment surface of a subject. Suitable gel materials for use with the ultrasound transducers of the ultrasound system are known in the art. Examples of suitable gel materials can include, without limitation, a gel, a gel-like composition, a hydrogel, a low density cross-linked polymer hydrogel, and the like. Suitable gels and hydrogels for use with the ultrasound transducer arrays of the present disclosure can include, without limitation, any gel or hydrogel effective to transfer ultrasound energy to a treatment area of a subject.

In certain embodiments, the plurality of ultrasound transducers are connected or secured by a woven or non-woven fabric to maintain form, and then connected with wiring.

In certain embodiments, the ultrasound transducer array is composed of a plurality of ultrasound transducers that are watertight, and are able to be immersed in water, gel, or hydrogel.

In certain embodiments, the ultrasound transducer array is immersed in a loaded hydrogel, which can be, without limitation, a mixture of therapeutic agents and hydrogel.

In certain embodiments, the ultrasound system further includes a securing component for keeping the ultrasound transducer array in place on a treatment surface of a subject.

In certain embodiments, the securing component can include, without limitation, a bandage, a wrap, an adhesive patch, a hydrogel coupling patch, or any other system for fixing the ultrasound transducer array in a desired location (e.g., a treatment area of the subject).

In certain embodiments, the securing component includes a hydrogel coupling patch and is connected to the mesh structure of the ultrasound transducer array by a clip component.

In certain embodiments, the hydrogel coupling patch includes an integrated non-woven adhesive and an ultrasound coupling agent.

In certain embodiments, the ultrasound system further includes an intermediate layer between the ultrasound transducer array and the securing component. More particular, in certain embodiments, the intermediate layer can include, without limitation, a foam layer.

In one embodiment, the ultrasound transducer system of the present disclosure includes a wound healing dressing integrated with an ultrasound transducer array of the present disclosure. In a particular embodiment, the wound healing dressing includes an ultrasound transducer array of the present disclosure having a flexible printed circuit connection. In certain embodiments, the wound healing dressing does not include any driving components for the ultrasound transducers. In certain embodiments, the wound healing dressing holds electronics and sensors. In certain embodiments, the wound healing dressing contains feedback thermal sensors for enhanced thermal regulation. In certain embodiments, the wound healing dressing contains conductive ink for sensors and the ultrasound transducers. In certain embodiments, the wound healing dressing is fully sterilizable. In certain embodiments, the wound healing dressing contains feedback sensors for ultrasound transducer and thermal sensor calibration.

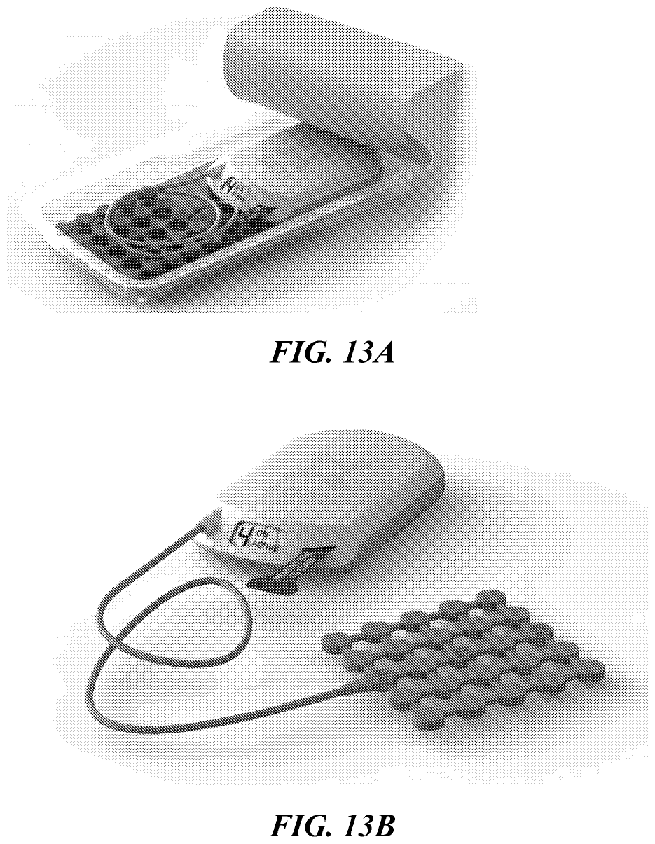

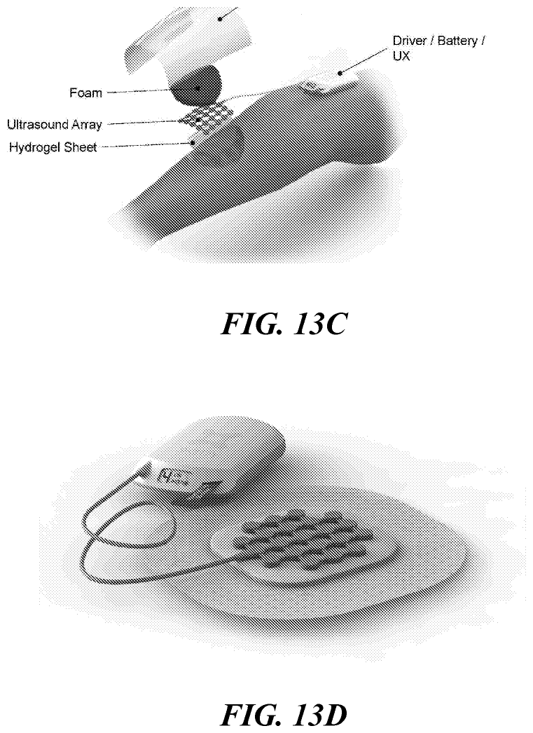

FIGS. 6 and 13A-13D illustrate various features of the ultrasound system of the present disclosure. With respect to FIGS. 6 and 13A-13D, the reference numbers of the ultrasound transducer system are identified in the paragraph below, as follows:

As shown in FIGS. 6 and 13A-13D, ultrasound system 100 includes: (i) ultrasound transducer array 110; and (ii) a power source 200 operably connected to ultrasound transducer array 110. In certain embodiments, ultrasound transducer array 110 is connected to power source 200 with a flexible cable 300. In certain embodiments, ultrasound system 100 further includes a securing component 400 for keeping ultrasound transducer array 110 in place on a treatment surface of a subject 500. In certain other embodiments, ultrasound system 100 further includes a hydrogel component 600 for placement between ultrasound transducer array 110 and a treatment surface of a subject 500. In certain other embodiments, ultrasound system 100 further includes an intermediate layer 700 (e.g., a foam layer) between ultrasound transducer array 110 and securing component 400.

In another aspect, the present disclosure provides a method of applying ultrasound energy to a surface of a subject. This method involves: (i) providing an ultrasound system according to the present disclosure; and (ii) applying therapeutic ultrasound energy to a subject, where the therapeutic ultrasound energy is generated by the ultrasound system.

In certain embodiments, the step of applying the therapeutic ultrasound energy to the subject includes the following steps: (i) securing the ultrasound transducer array to a treatment area of the subject; and (ii) operating the ultrasound system under conditions effective to apply the therapeutic ultrasound energy to the treatment area of the subject.

In certain embodiments of the method of the present disclosure, the ultrasound transducer array is compressed to the treatment area of a subject with a gel or hydrogel pad.

In certain embodiments of the method of the present disclosure, the ultrasound transducer array is fixed to the treatment area of a subject with an adhesive bandage.

In certain embodiments of the method of the present disclosure, the ultrasound transducer array is fixed to the treatment area of the subject with a fabric wrap.

In certain embodiments, the ultrasound transducer array is configured for use to deliver ultrasound energy for a medical application that can be used with ultrasound energy.

In certain embodiments, the ultrasound transducer array is used for treating a musculoskeletal injury or to accelerate repair of a musculoskeletal injury.

In certain embodiments of the method of the present invention, a hydrogel is used to couple the ultrasound transducer array to the treatment area of a subject. In such embodiments, adhesive bandages can be used to hold the ultrasound transducer array firmly to the skin of the subject. In these embodiments, a flexible ultrasound transducer array can be integrated with an ultrasound coupling agent connected directly to the treatment area of the subject and formed to the contours of the skin of the subject. In certain embodiments, the ultrasound transducer array is permeable, thereby allowing the flow of exudates out of a wound site or application area, which allows and facilitates the use of the ultrasound transducer array and system of the present disclosure for long-term wear.

In certain embodiments, applying the therapeutic ultrasound energy to the treatment area is effective to alleviate pain in tissue of the subject in and around the treatment area.

In certain embodiments, applying the therapeutic ultrasound energy to the treatment area is effective to heal soft tissue of the subject in and around the treatment area.

In certain embodiments, applying the therapeutic ultrasound energy to the treatment area is effective to heal wounding of tissue of the subject in and around the treatment area.

In certain embodiments, the method further involves delivering a drug to the treatment area of the subject with the therapeutic ultrasound energy.

In certain embodiments, the method further involves conforming the ultrasound transducer array to the contour of the treatment area of the subject. More particularly, in certain embodiments, the step of conforming involves removing one or more of the ultrasound transducers from the area in order to adequately cover and/or form-fit the treatment area with the ultrasound transducer array. In certain embodiments, the step of conforming involves manipulating the ultrasound transducer array to conform to the contour of the treatment area.

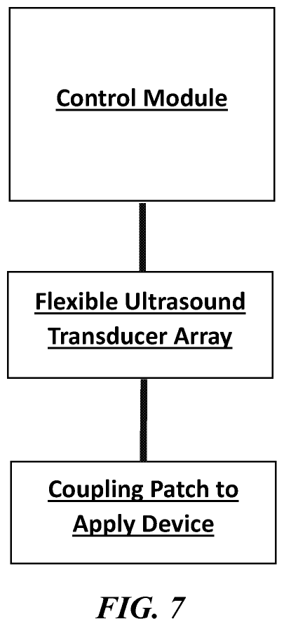

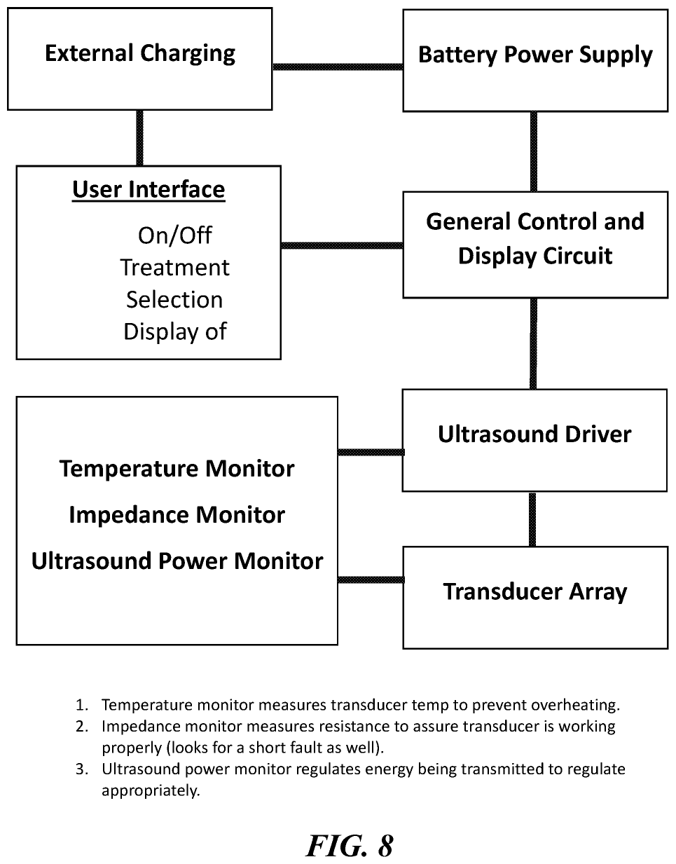

FIGS. 7 and 8 are schematics showing components of the ultrasound transducer array and system functionality and connectivity.

### EXAMPLES

The following examples are intended to illustrate particular embodiments of the present invention, but are by no means intended to limit the scope of the present invention.

### Example 1

Various embodiments of the ultrasound transducer array and ultrasound system of the present disclosure have been constructed and tested for various attributes and applications. Provide below are examples of certain of these ultrasound transducer arrays and ultrasound systems.

In one exemplary embodiment, the ultrasound transducer array of the present disclosure is powered by an external power device and secured to a subject/patient (e.g., human or animal) via an adhesive bandage. Hydrogel is used to couple the ultrasound transducer array to the treatment tissue area of the subject. The ultrasound transducers emit energy continuously into the tissues, which accelerates healing of wounds and injuries. Further, the device may be used to deliver drugs to tissue. The flexibility of the ultrasound transducer array allows for long-term wear by the subject without creating a pressure spot.

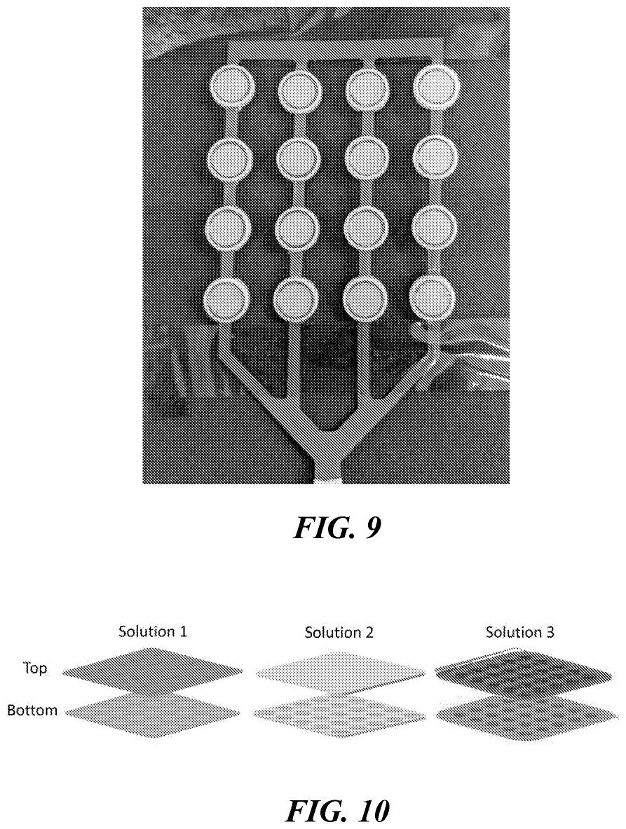

FIG. 9 illustrates one exemplary embodiment of an ultrasound transducer array of the present disclosure. As shown in FIG. 9, the ultrasound transducer array is a flexible ultrasound transducer array. The bottom side (i.e., patient contact side) of the transducer array is shown in FIG. 9. Ultrasound emitted from each transducer of the array is transmitted into the tissue via an ultrasound coupling agent. Each transducer may have a lens for focusing or dispersion of the ultrasonic wave, and a variety of different types of lenses may be used for creating ultrasound field patterns in the tissue. Another feature is the redundancy for the design with transducers and electrical connections, which allows for the transducer array to be cut into various forms (post-fabrication) to fit a corresponding geometry or contour on the patient and still function appropriately (e.g., like a sheet of paper that can be cut).

An ultrasound transducer power pack has been constructed for testing. An exemplary embodiment of the power pack was constructed to control the on/off functionality of the transducer array and be supported by internal batteries. This power pack provides control of electrical energy to excite the flexible wearable ultrasound transducer. The power pack provides timing, treatment logging, dossing measurements, power-output, and confirms transducer type to be connected to the power pack.

Hydrogel is used to couple the transducer array to the treatment area. Adhesive bandages hold the transducer array firmly to the skin. In this embodiment, the flexible transducer is integrated with an ultrasound coupling agent connected directly to the treatment area and contours to the skin. The transducer is permeable allowing the flow of exudates out of the wound site or application area to allow for long-term wear.

Various embodiments of transducer arrays for the flexible transducer/ultrasonic dressings and associated driving electronics have been developed for testing of ultrasound delivery and form factors.

In constructing the ultrasound transducer arrays and ultrasound systems of the present disclosure, various features were included. Some of these features included arrays and systems having the following features: (i) patient contacting surfaces are sterilizable; (ii) patient non-contacting surfaces allow for disinfection; (iii) arrays and systems that are flexible, conformable, and low-profile; (iv) arrays and systems that have even pressure distribution over a large treatment area to prevent pressure ulcers; (v) flexible transducer arrays and systems that can treat and interface with a variety of shapes, sizes, and locations on the body of the subject; (vi) transducer arrays that are disposable, low-cost, and easily applied; (vii) transducer arrays and systems that allow secure coupling to skin and allow mobility of the subject; and (viii) a treatment control module that allows programmable treatment regimens by the condition being treated.

During development of the ultrasound transducer arrays and ultrasound systems of the present disclosure, various designs were produced. FIG. 10 illustrates three of these designs, which are further described in Table 1 below:

TABLE 1

| | | Clinical/ | |
|---|---|---|---|
| | | Commercial | Technical |
| Name | Description | Advantages | Requirements |
| Solution 1: | Transducer | Lowest-Profile | Attachment of dressing |
| Laminated | array connected | Lightest | to skin |
| Matrix | via flex circuit | Most Flexible | Isotropic flexibility of |
| | and laminated | Disposable | flex circuit |
| | between two | Lowest | Piezoceramic binding to |
| | waterproof, | Manufacturing | flexible substrate. |
| | flexible sheets | Cost/Fewest | |
| | | Components | |
| Solution 2: | Transducer | Low-Profile | Maintaining seals at |
| Silicone | array embedded | Flexible | multiple component |
| Backed | in a silicone | Disposable | interfaces. Isotropic |
| Matrix | structure and | | flexibility of flex circuit. |
| | connected via | | |
| | flex circuit | | |
| Solution 3: | Transducer | Semi-Flexible | Minimizing profile and |
| Wired | array embedded | | weight. |
| Matrix | in silicone and | | Minimizing cost due to |
| | connected with | | extensive use of wires |
| | wires | | and manual assembly. |

### Example 2

### Construction and Testing of an Ultrasound Transducer Array and System

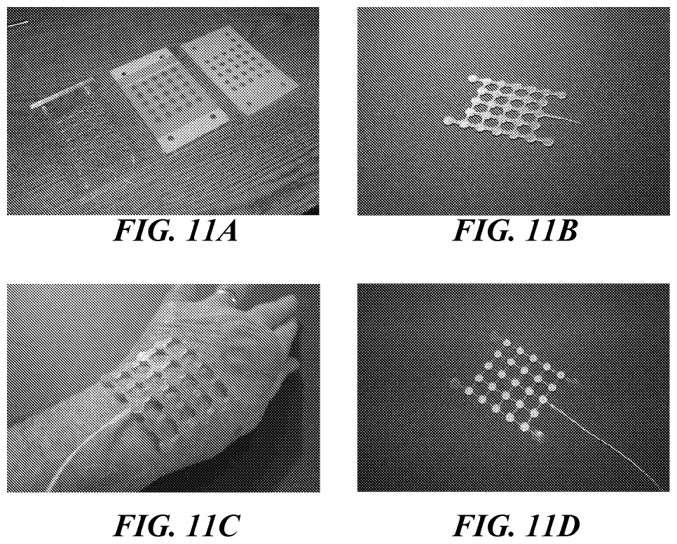

In one exemplary embodiment, there was constructed a 25 element (transducer), 3 MHz flexible ultrasound transducer array cast in polyurethane with silver/tin electrical connections. The array was constructed to have redundant wiring to allow for custom trimming when applied. The construction process of the 5×5 array of 6 mm transducer elements first consisted of developing a CNC mold matrix (see FIG. 11A) to cast the flexible polyurethane outer housing to secure the active elements. As shown in FIG. 11A and FIG. 11C, the orange colored disks, in the center, are active transducers and flat ribbon conductors run between them in one direction, joined at the bottom by a cross ribbon conductor.

As shown in FIGS. 11A-11D, the 4 outer disks on each corner of the mold are used to remove the transducer from the mold and may be cut off. As shown in FIG. 11C, a coaxial cable is connected to the input of the transducer array and exits the matrix at the tail section in the middle outside of the matrix. Another array was also produced using silver active elements. Individual electrical connections where made with press fit and soldering approaches. Soldering proved more robust and provided better electrical conductivity.

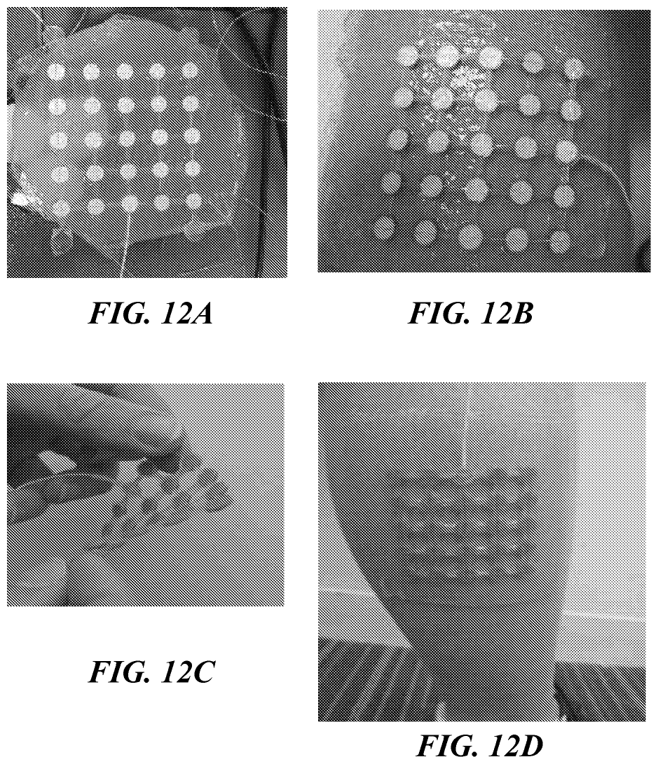

Testing was conducted of high-capacitance driving stages used to power the ultrasonic flexible transducer. A 3.7V ultralow impedance booster/mosfet architecture was effective for powering the flexible wearable ultrasound transducer array. The ultrasound transducer testing included acoustic, electrical, and ergonomic testing. FIGS. 12A-12D illustrate certain embodiments of the ultrasound transducer array and system used in the testing.

Flexible ultrasound transducer arrays performed as specified over the 4-12 hour testing intervals with sustained electrical drive and ultrasonic production across the 25-100 element devices. Power handling requirements were 0.35 amps at 6 volts (2.1 Watts) and 0.12 amps at 3 volts (0.36 Watts).

Continuous testing of an optimized array was completed with sustained delivery of ultrasound for 7 continuous days. Silicone coating prevented delamination and oxidization of any of the devices' electrical features. Electrical power and acoustic energy was held constant for the entire study duration.

A 25-transducer array was studied under functional testing post porcine treatment study. All aspects of the array were found to remain functional post-treatment and cleaning.

Various flexible circuit and design concepts have gone through multiple iterations to allow for construction of long-duration sustained acoustic medicine flexible ultrasound transducer arrays. For prototyping, a suitable ultrasound transducer array was found to be 5.0×5.0 cm and 0.2 cm thick, with a power pack of 5.0×5.0 cm and 1 cm thick.

One design for a commercial SAM® (sustained acoustic medicine) flexible ultrasound array and system was developed, as shown in 3D printing in FIGS. 13A-13D. 3D models and rapid prototyping was used in the process of prototype construction. FIG. 13A shows the ultrasound system in sterile packaging. FIG. 13B shows the ultrasound system with the power source/controller having an activation pull-tab. FIG. 13C shows the ultrasound system being used to apply ultrasound treatment to a patient. FIG. 13D shows the ultrasound system in a configuration where the ultrasound transducer array is used with a bandage. As shown in FIG. 13D, the ultrasound system is used as a SAM® wound healing device, with the ultrasound transducer array being attached to a foam pad and an adhesive patch being used for application to a wound.

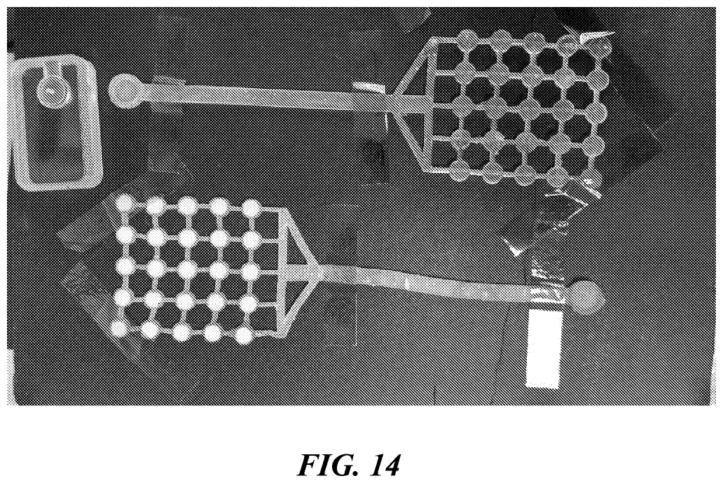

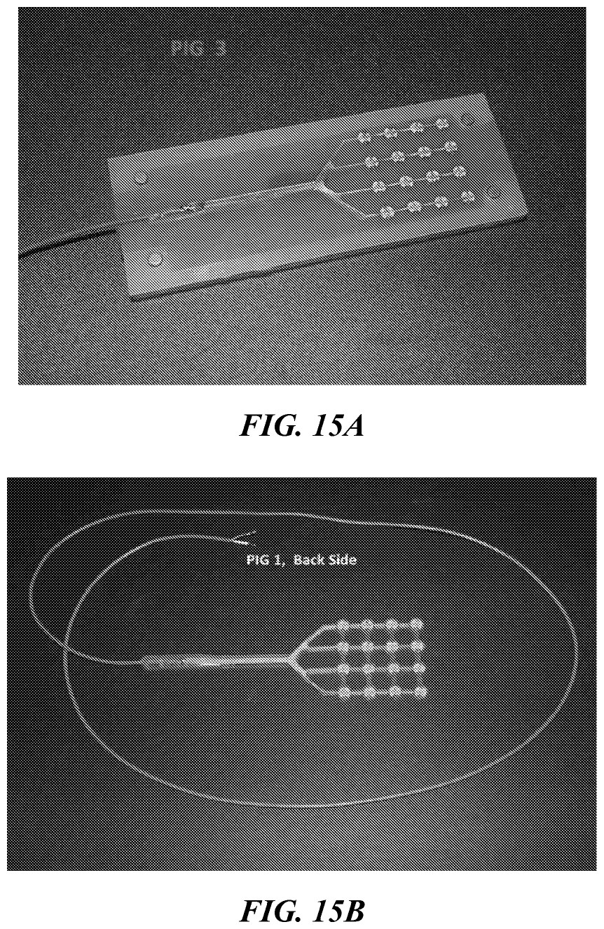

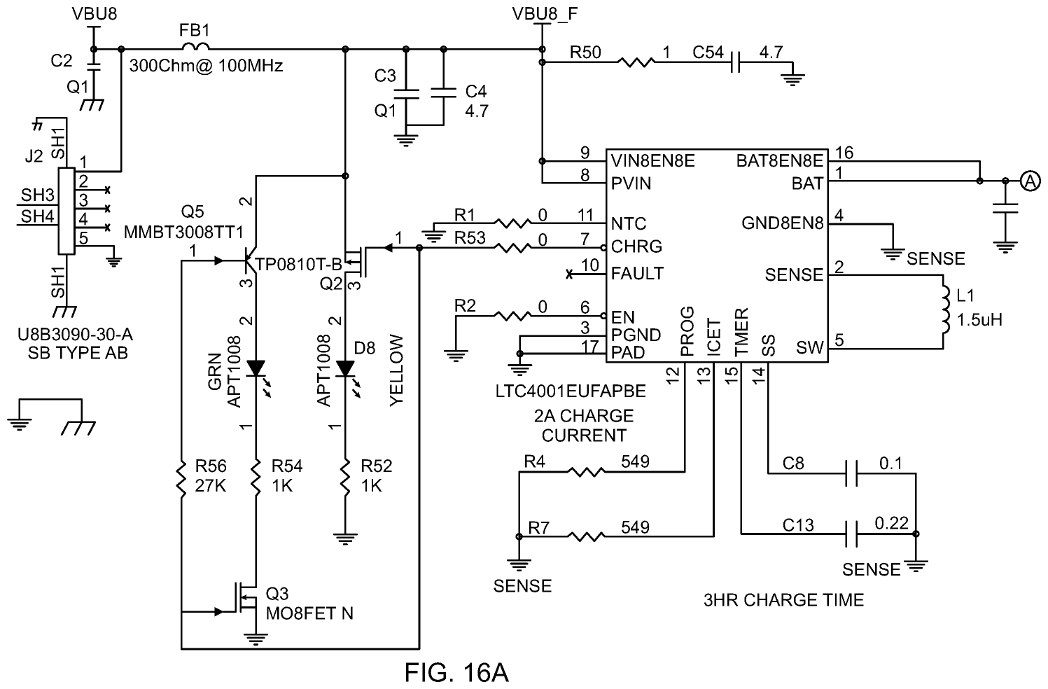

In certain embodiments of the ultrasound transducer array and system that were tested, the ultrasound transducer array construction may be completed with wrap-tab piezo materials, as shown in FIG. 14.

For flexible transducer arrays, both large and small transducer arrays have been constructed with new custom-flex PCBAs and evaluated on the lab bench (see FIGS. 15A-15B). FIG. 15A illustrates an ultra flexible array (16×16) before over mold to encapsulate electrical connections. FIG. 15B illustrates the ultra flexible array (16×16) having an over mold double-copper array with medical-silicone.

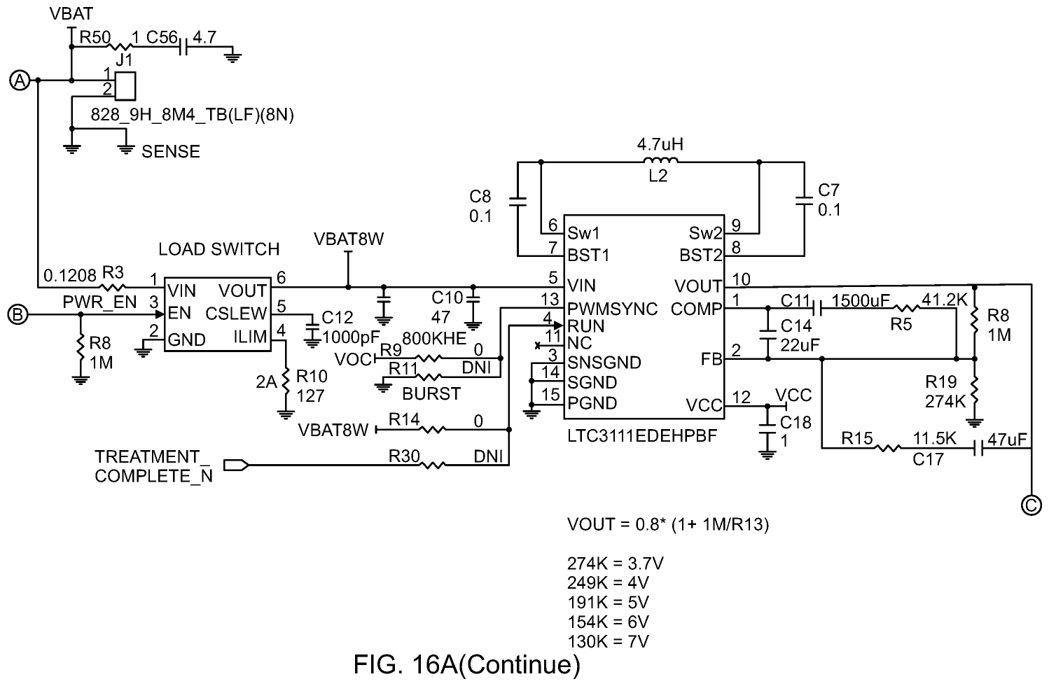

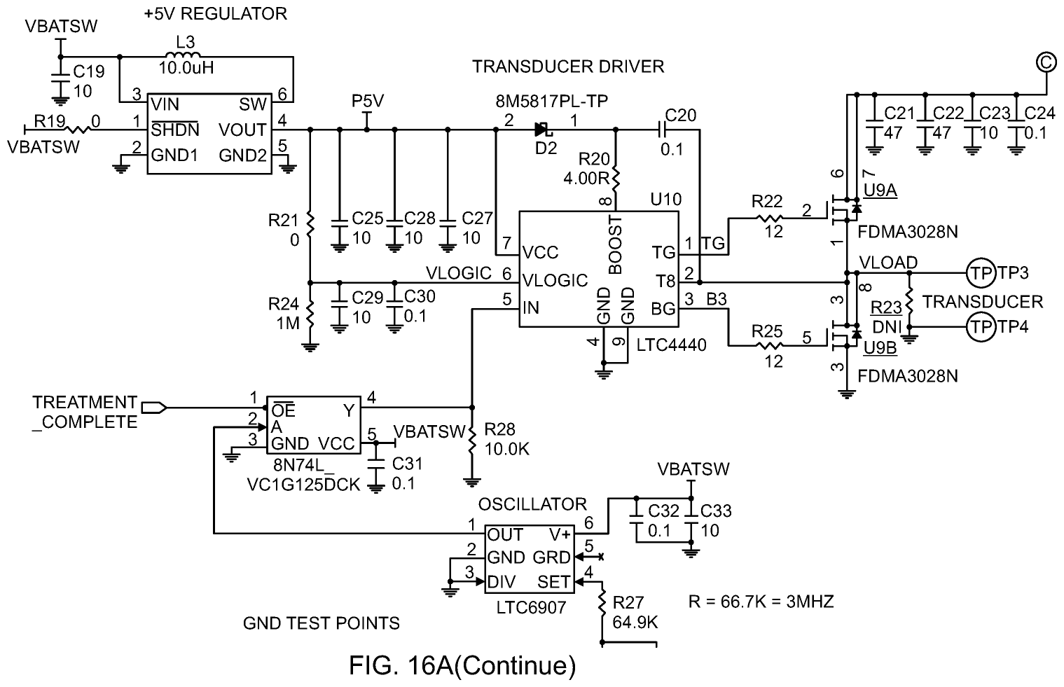

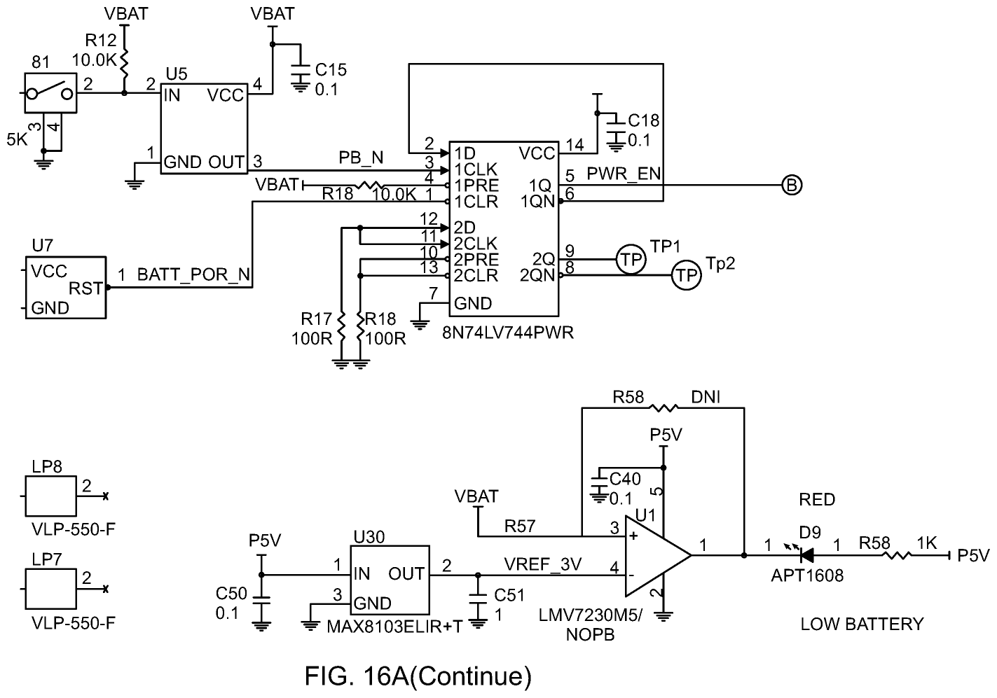

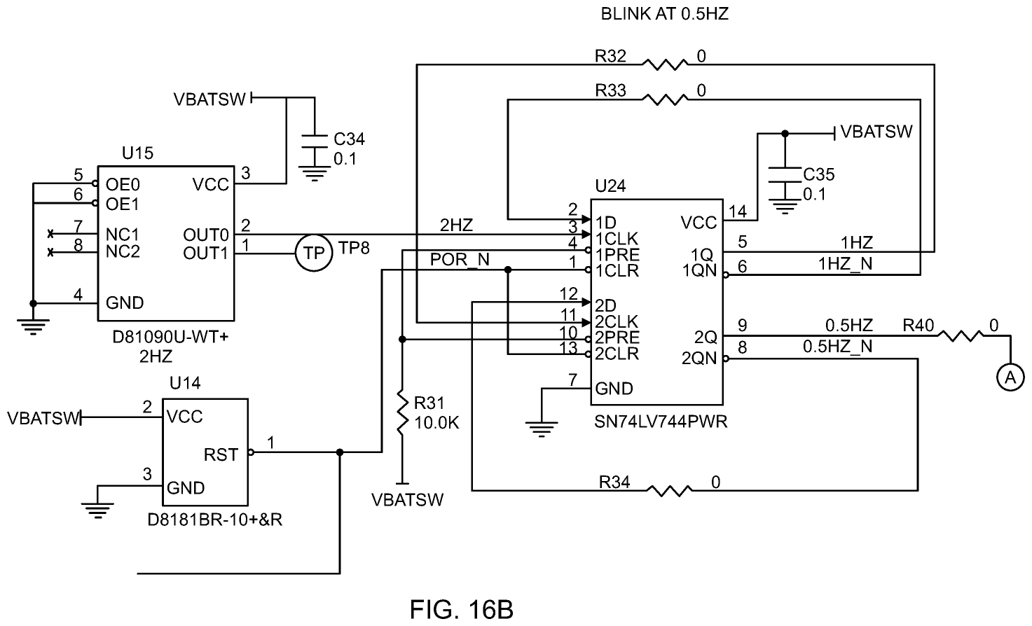

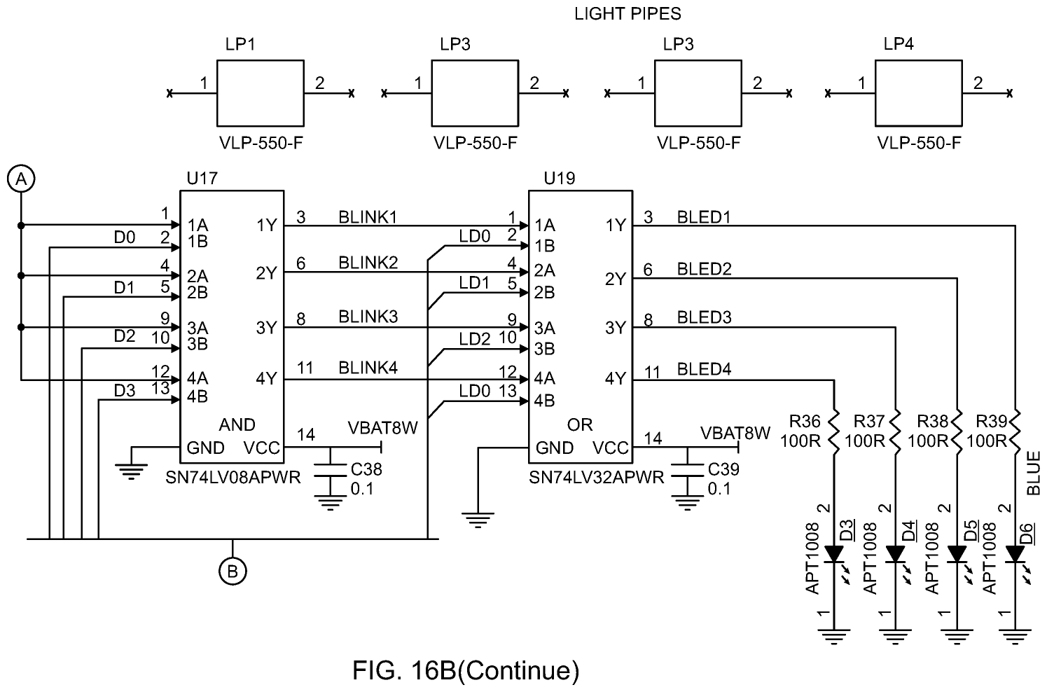

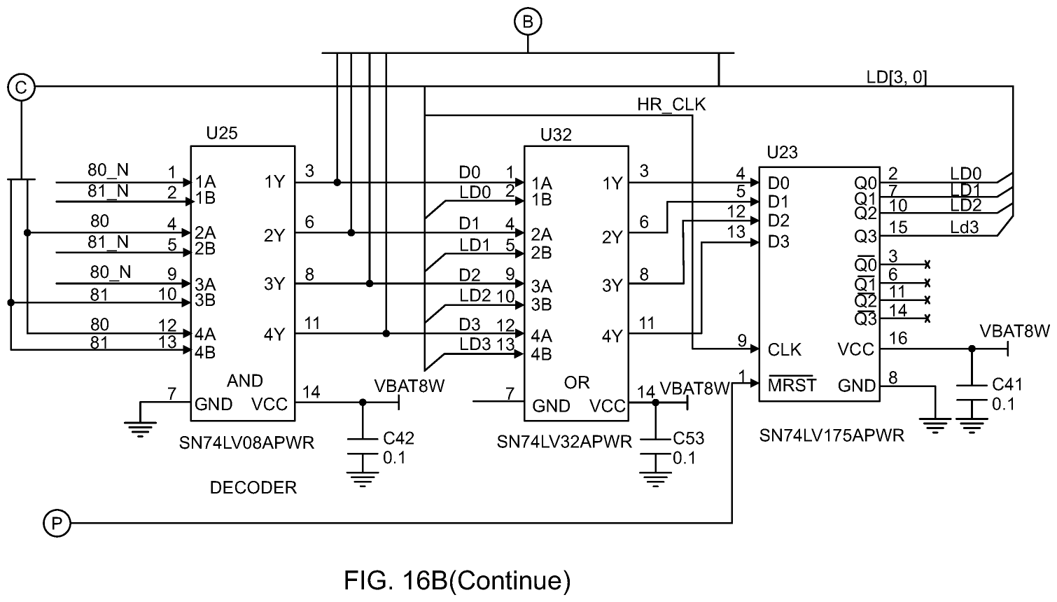

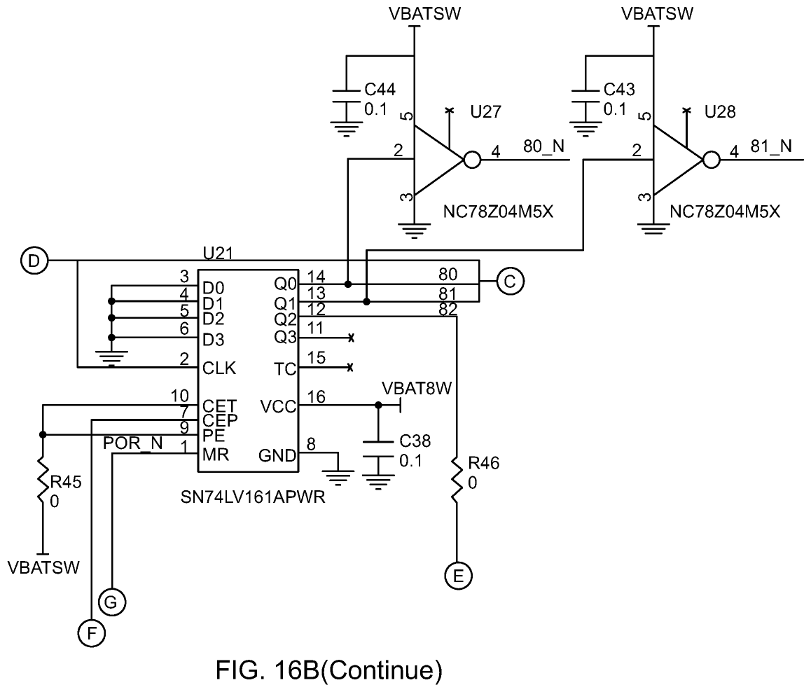

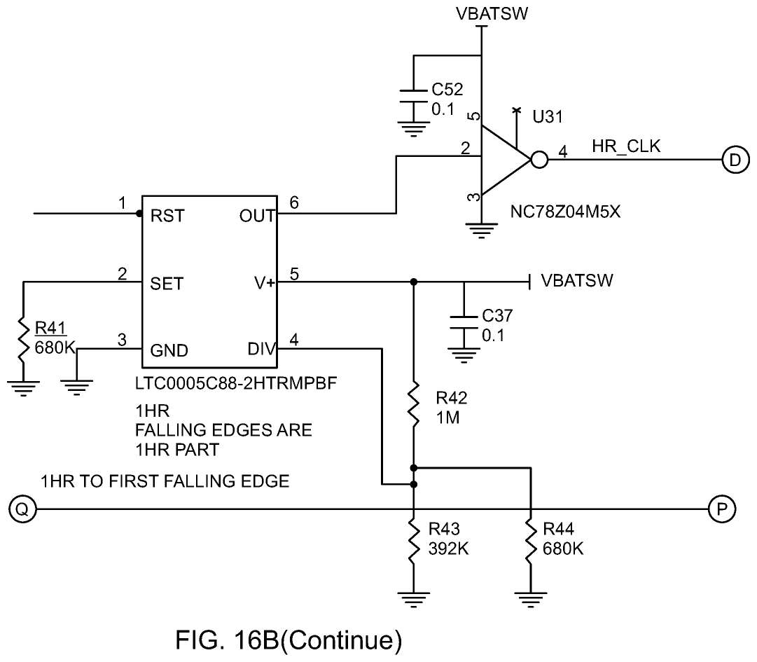

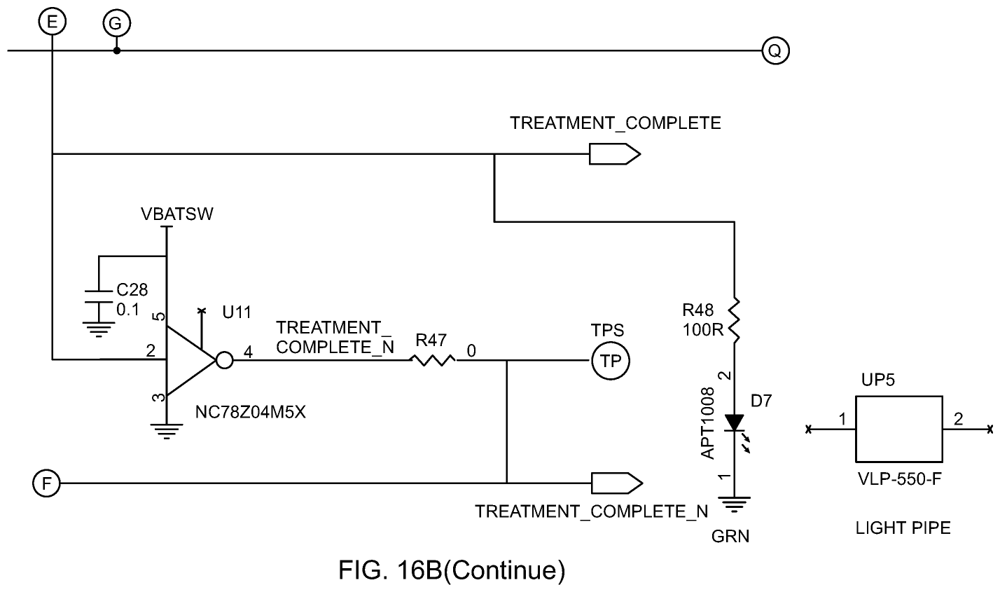

Various embodiments of the electronics for driving the ultrasound transducer array were constructed and tested. Circuit diagrams were completed to support final PCBA layout into the controller housing. One embodiment included circuitry as shown in the circuit diagrams of FIGS. 16A and 16B. The circuit diagrams of FIGS. 16A and 16B show a design used for animal testing of the ultrasound transducer array and system. Briefly, the design provides treatment timing features, ultrasound power control, single-use and recharge capability, manufacturing frequency/voltage adjustment for calibration, and on/off activation of the device. FIG. 16A is a circuit diagram for an embodiment of the ultrasound transducer array and system for use as a flexible ultrasound transducer driver device. FIG. 16B is a circuit diagram for an embodiment of the ultrasound transducer array and system for use as a wound healing device.

### Example 3

### Exemplary Flexible and Semi-Rigid Ultrasound Transducer Arrays and Systems

Various embodiments of flexible and semi-rigid ultrasound transducer arrays and systems have been prepared for testing.

FIG. 17 illustrates one exemplary embodiment of a SAM® flexible ultrasound transducer array and system having a hydrogel dressing attached to transducer array.

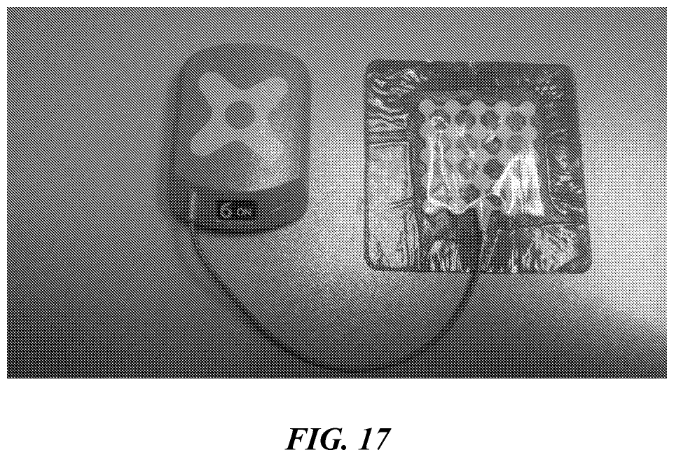

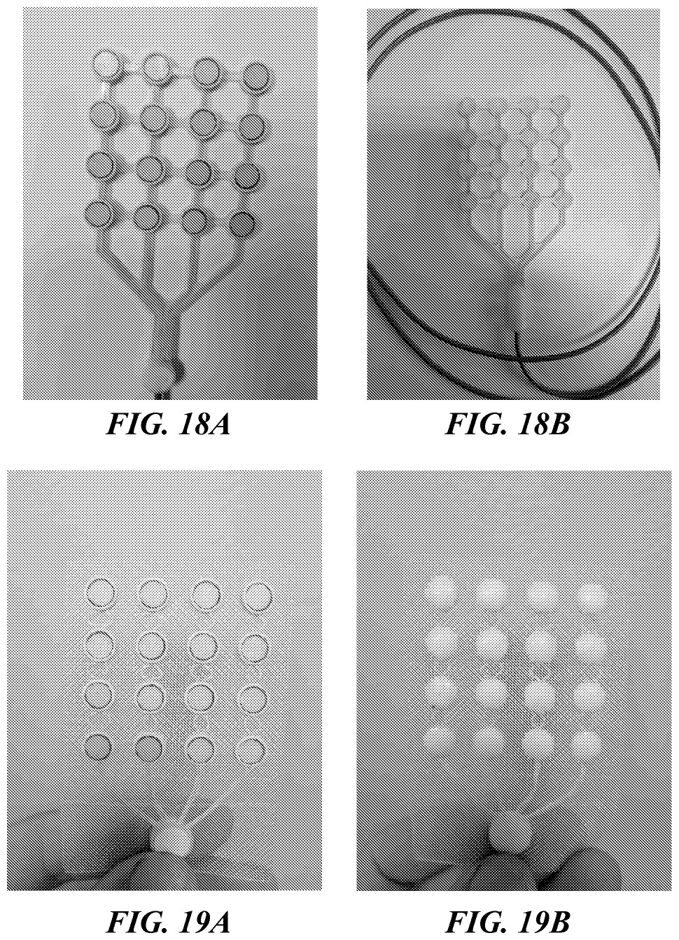

Additional flexible ultrasound transducer array designs where developed and manufactured during the ruggedization process for testing as shown in FIGS. 18A, 18B, 19A, and 19B.

As shown in FIGS. 18A and 18B, there is illustrated a semi-ridged case to house wires and transducers. The semi-rigid wound healing transducer array is shown with a blunted end. The flex PCB is encased in a flexible epoxy and each individual transducer element is housed in a unique cylindrical housing with foam backing. The design reduces water egress between transducer/pcb/housing interfaces.

As shown in FIGS. 19A and 19B, there is illustrated an ultra-flexible screen transducer with embedded transducer elements and flexible cables. The ultra-flexible transducer array is shown with a blunted end. Each transducer element is individually housed and wired with flexible electrical cable. Each element is aligned and secured into a flexible nylon mesh to provide mechanical strength. The wires of each transducer element are knitted into the mesh for durability.

The ultrasound transducer array shown if FIGS. 19A-19B provides an excellent platform for encasing in hydrogel.

Additional specifications of an ultrasound transducer array and system exemplified by FIGS. 19A-19B can include, without limitation, the following:

The design includes a mechanical mesh material (e.g., nylon, metal, polymer, silicone etc.) that acts as the mechanical support for the flexible ultrasound transducer array.

Electrical conductivity is provided by a redundant wiring network (wires, coaxial-cable, flexible circuit) that has additional relief to bend/contour to any shape that is limited by the mechanical mesh material mentioned above.

The transducer array is made up of individual ultrasound transducers that are independent and isolated so that each element may work by itself or in unison. The transducer elements may be single crystal type in the shape of disk, rectangle, triangle, square or other. The transducer elements may be multiple crystal design in the form of stacks to provide low electrical impedance and excitation voltage for the element. The transducer elements may have convex, concave or flat lenses made from TPX, Ultem, Rexolite, metal, or other materials sufficient to protect the element and isolate the transducer. The transducer elements are air-backed with thermocouple embedded into housing to monitor temperature of transducer.

The transducer elements are positioned into a matrix and connected electrically via wire or flexible circuit and sealed to prevent any electrical shorting. The electrical/transducer array is then secured to the mechanical mesh via epoxy, glue, or other connecting medium to fabricate a transducer assembly. The act of securing the transducer element to the mechanical mesh can also provide a back-seal to the transducer element that can be accomplished with a mixture of epoxy and microbubbles. This forces the sound to go into the patient versus being absorbed in the ultrasound device itself.

In one embodiment, a hydrogel will be coated onto the front face of the transducer array and mesh to provide ultrasonic coupling between the array and patient contacting surface.

One important feature is the attachment of the ultrasound transducer array to the patient. Since attachment is important, additional features of the ultrasound transducer array and system of the present disclosure include, without limitation: (i) the flexible transducer array may self-adhere to the body with hydrogel coupling; (ii) the flexible transducer array may be covered by a bandage, wrap, etc. to secure in place; (iii) the flexible transducer array may clip into a hydrogel coupling patch that secures and couples the device to the body. This clipping is accomplished by a clip feature on the mesh-structure of the ultrasound transducer array interfacing with an ultrasound coupling patch with integrated non-woven adhesive and ultrasound coupling agent. The ultrasound coupling patch could be disposable. This concept would be similar to SAM® devices available in the art currently, but with a flexible ultrasound transducer array of the present disclosure.

Functional testing of exemplary ultrasound transducer arrays and power controllers were successful post animal study.

The flexible ultrasound transducer array that was developed and tested has successfully demonstrated sustained acoustic medicine delivery in vivo as designed.

Assembly was completed of an ultra-flexible ultrasound transducer for final device testing, and in process of completing flexible PCBA-based transducers for laboratory testing and final commercial prototype study. Eight ultra-flexible ultrasound transducer arrays fabricated for animal study were produced based off of study performance results. Each ultrasound transducer array will be powered by a controller system.

During the construction of the flexible-PCBA transducer array, the insulation was removed mechanically from the PCBA to improve electrical conductivity and the inner-ring and outer-ring compartments that secure to the nylon mesh were revised. This will allow the flexible PCBA to nest into the air-backed housing and maintain electrical/mechanical integrity.

The process of assembly of the ultra-flexible transducer merged with the flexible-PCBA involved construction of encapsulation housings. The encapsulation housing are aligned on a fixture with flexible PCBA, soldered and then backed with glue/bubbles to secure to the nylon mesh.

Animal Testing: Animal evaluation and analysis of the flexible ultrasound transducer in third degree burn wounds were completed. Briefly, the device was applied to the porcine wound with hydrogel dressing and bandage. Each ultra-flexible ultrasound transducer array was powered/calibrated to two power controller modules. The treatment groups included a group using an active commercial prototype device of the ultrasound transducer array and a group using a placebo device.

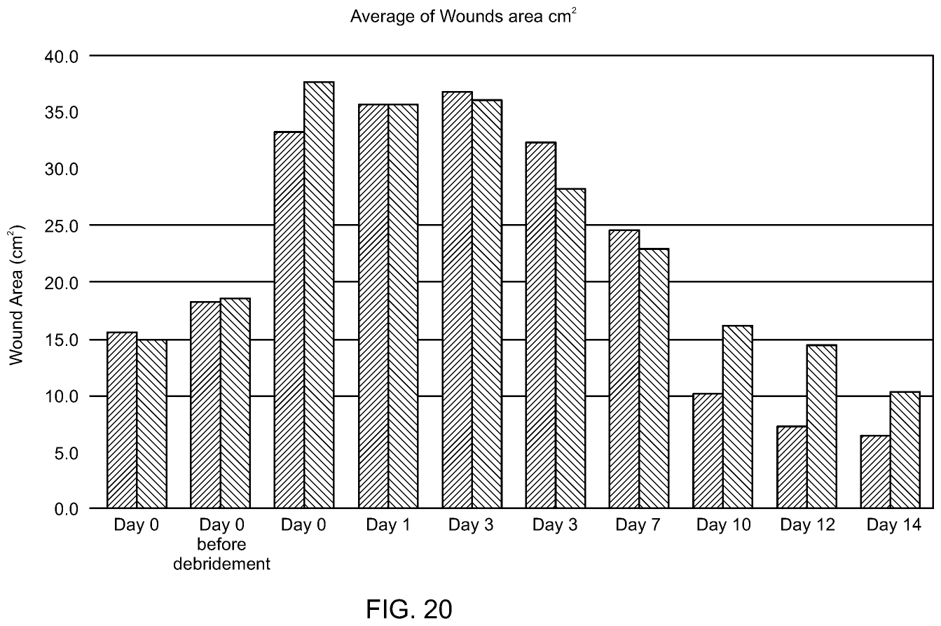

Animal experiments were conducted using the commercial prototype in burn wounds with seven active and seven placebo devices. The results from daily treatment of the active ultrasound device showed 43% reduced wound size in day 10, 12, and 14 assessment points p<0.05. This data demonstrates that the device is healing/closing the wound at an accelerated rate. FIG. 20 is a graph showing wound closure of the wound healing device compared with the placebo device over 14 days.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to patents and printed publications throughout this specification. Citation of a reference herein shall not be construed as an admission that such reference is prior art to the present invention. All references cited herein are hereby incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Although the present invention has been described for the purpose of illustration, it is understood that such detail is solely for that purpose and variations can be made by those skilled in the art without departing from the spirit and scope of the invention which is defined by the following claims.

1. An ultrasound transducer array comprising:
a. a plurality of ultrasound transducers arranged in a matrix formation and operably coupled to an electrical network; and
b. a mesh structure securing the plurality of ultrasound transducers in the matrix formation,
wherein each ultrasound transducer is connected to the electrical network in a manner sufficient to allow each ultrasound transducer to operate independent of one another or in unison.

2. The ultrasound transducer array according to claim 1, wherein the array is configured to be flexible and wearable, thereby being suitable for long duration ultrasound application to a subject.

3. The ultrasound transducer array according to claim 1, wherein said matrix formation comprises the plurality of ultrasound transducers oriented in substantially the same plane with their ultrasound emitting surfaces facing the same direction.

4. (canceled)

5. The ultrasound transducer array according to claim 1, wherein said electrical network is configured to have redundant wiring.

6. (canceled)

7. The ultrasound transducer array according to claim 1, wherein said electrical network is configured to have redundant wiring with each transducer in parallel to minimize electrical impedance of the transducer array.

8. (canceled)

9. (canceled)

10. The ultrasound transducer array according to claim 1, wherein said electrical network comprises electrical components selected from the group consisting of electrical wires, coaxial cable, flexible printed circuit board (PCB), and a flexible circuit, or combinations thereof.

11. (canceled)

12. The ultrasound transducer array according to claim 1, wherein the mesh structure comprises a mechanical mesh material selected from the group consisting of nylon, metal, polymer, silicone, plastic, fibers, and a plant-derived compound, or a combination thereof.

13. (canceled)

14. The ultrasound transducer array according to claim 1, wherein the ultrasound transducers are configured as low-profile ultrasound transducers comprising one or more of the following attributes:
a. a frequency of between about 250 kHz to about 4 MHz;
b. a thickness of less than 1 cm; and/or
c. power output capability of 0-3 Watts, and intensity of 20 mW/cm²-10 W/cm².

15. (canceled)

16. The ultrasound transducer array according to claim 1, wherein the ultrasound transducer array has an electrical input impedance of less than 1 ohm, less than 0.25 ohm, or less than 0.01 ohm.

17. The ultrasound transducer array according to claim 1, wherein the ultrasound transducers are single crystal type transducers.

18. (canceled)

19. (canceled)

20. The ultrasound transducer array according to claim 1, wherein the ultrasound transducers are multiple crystal design transducers.

21. The ultrasound transducer array according to claim 20, wherein the ultrasound transducers are multiple crystal design transducers comprising piezoelectric stacks in parallel for low-frequency and low electrical impedance for ultrasound power production in a flexible form.

22. (canceled)

23. The ultrasound transducer array according to claim 1, wherein the ultrasound transducers further comprise a lens selected from the group consisting of a convex lens, a concave lens, and a flat lens.

24. (canceled)

25. The ultrasound transducer array according to claim 1, wherein the ultrasound transducers are air-backed with a thermocouple embedded into a housing sufficient to enable monitoring of the temperature of the ultrasound transducers during operation.

26. (canceled)

27. The ultrasound transducer array according to claim 20, wherein the ultrasound transducers and electrical network are secured to the mesh structure with a connection medium and or method selected from the group consisting of epoxy, glue, welding, magnetic, adhesive tape, and compression fit.

28. The ultrasound transducer array according to claim 1, wherein the ultrasound transducers are secured to the mesh structure by a back-seal comprising a mixture of epoxy and microbubbles.

29. The ultrasound transducer array according to claim 1 further comprising:
a hydrogel coated to the ultrasound emitting face of the ultrasound transducer array and mesh structure to enable ultrasonic coupling between the array and a subject contacting surface.

30. An ultrasound system comprising:
a. an ultrasound transducer array according to claim 1; and
b. a power source operably connected to the ultrasound transducer array.

31. (canceled)

32. (canceled)

33. (canceled)

34. (canceled)

35. The ultrasound system according to claim 30 further comprising:
a securing component for keeping the ultrasound transducer array in place on a treatment surface of a subject.

36. (canceled)

37. The ultrasound system according to claim 35, wherein the securing component comprises a hydrogel coupling patch and is connected to the mesh structure of the ultrasound transducer array by a clip component.

38. The ultrasound system according to claim 37, wherein said hydrogel coupling patch comprises an integrated non-woven adhesive and an ultrasound coupling agent.

39-49. (canceled)
